(19) United States
(12) Patent Application Publication
(10) Pub. No.: US 2023/0197247 A1
(43) Pub. Date: Jun. 22, 2023

(54) RADIOGRAPHIC INTERPRETATION MANAGEMENT APPARATUS, STORAGE MEDIUM, AND RADIOGRAPHIC INTERPRETATION MANAGEMENT METHOD

(71) Applicant: Konica Minolta, Inc., Tokyo (JP)

(72) Inventors: Hiroshi TERAO, Tokyo (JP); Ken SATOU, Tokyo (JP); Shinsuke KATSUHARA, Tokyo (JP)

(21) Appl. No.: 18/081,773

(22) Filed: Dec. 15, 2022

(30) Foreign Application Priority Data
Dec. 20, 2021 (JP) 2021-205717

(51) Int. Cl. G16H 30/20; G06T 7/00

(52) U.S. Cl. CPC G16H 30/20; G06T 7/0012

(57) ABSTRACT

A radiographic interpretation management apparatus includes a hardware processor. The hardware processor is configured to obtain an automatically generated finding obtained by computer processing on medical information, obtain a radiographic interpretation finding created by a user based on the medical information, and be able to set by user operation a combination that is to be displayed with highlight among combinations of a result of the automatically generated finding and a result of the radiographic interpretation finding.

### CROSS REFERENCE TO PRIOR APPLICATIONS

The present invention claims priority under 35 U.S.C. § 119 to Japanese Patent Application No. 2021-205717 filed on Dec. 20, 2021, the entire content of which is incorporated herein by reference.

### TECHNICAL FIELD

The present invention relates to a radiographic interpretation management apparatus, a storage medium, and a radiographic interpretation management method.

### DESCRIPTION OF THE RELATED ART

Lately, with the development of artificial intelligence (AI) technology, analysis by AI is introduced in the medical field. Attempts are made to support with AI the analysis and diagnosis of medical information such as image diagnosis that were conventionally performed by doctors.

In the clinical practice of medicine, there is a need to perform testing and diagnosis suitably and rapidly in order to perform diagnosis efficiently and to optimize the diagnosis so that the burden on the doctor can be reduced.

The introduction of the AI analysis is expected to contribute to the efficiency and the optimization of such diagnosis.

For example JP 2009-86750 describes analysis result information in which information related to the field of medicine extracted from a radiographic interpretation report created by the user is associated with items compared with diagnosis support information in which information related to the field of medicine obtained from the medical examination data is associated with items. According to the comparison, the difference for each item is detected, and a degree of importance is determined for the difference detected for each item. Then, the manner of display showing the detected difference is changed according to the determined degree of importance and this is presented with the display of the contents of the radiographic interpretation report information. An apparatus with the above function is disclosed.

### SUMMARY OF THE INVENTION

However, in the combination of the radiographic interpretation result by the doctor and the AI radiographic interpretation result (contrast result), the contrast result that the medical facility or the user desires to display with highlight may differ depending on the medical facility or the user. For example, when the contrast result is confirmed by the second radiographic interpretation doctor, there are cases such as when the second radiographic interpretation doctor desires to confirm only when the radiographic interpretation result by the doctor (first radiographic interpretation doctor) is different from the interpretation result by the AI, when the second radiographic interpretation doctor desires to confirm only when there is a possibility that the first radiographic interpretation doctor overlooked a lesion, or when the second radiographic interpretation doctor desires to confirm only when there is a possibility that both the first radiographic interpretation doctor and the AI overlooked a lesion. There is a need to display the contrast result with highlight according to the situation that the doctor desires to confirm as described in the examples above.

According to the invention described in JP 2009-86750, the display with highlight desired by various medical facilities and users cannot be achieved when the needs are different depending on the medical facility or the user.

As described above, the highlighted display desired when the radiographic interpretation is performed is different depending on the medical facility, for example. However, it is costly and time-consuming to develop and manufacture the apparatus (radiographic interpretation management apparatus, analysis apparatus) to achieve each type of desired highlighted display.

The present invention is conceived in view of the above problems, and the object of the present invention is to provide a radiographic interpretation management apparatus, storage medium, and radiographic interpretation management method that can achieve the desired highlighted display when the radiographic interpretation is performed.

To achieve at least one of the abovementioned objects, according to an aspect of the present invention, a radiographic interpretation management apparatus reflection one aspect of the present invention includes a hardware processor, wherein the hardware processor is configured to, obtain an automatically generated finding obtained by computer processing on medical information, obtain a radiographic interpretation finding created by a user based on the medical information, and be able to set by user operation a combination that is to be displayed with highlight among combinations of a result of the automatically generated finding and a result of the radiographic interpretation finding.

According to another aspect, anon-transitory computer-readable storage medium storing a program causing a computer to perform: first obtaining to obtain an automatically generated finding obtained by computer processing on medical information, second obtaining to obtain a radiographic interpretation finding created by a user based on the medical information, and setting to be able to set by user operation a combination that is to be displayed with highlight among combinations of a result of the automatically generated finding and a result of the radiographic interpretation finding.

According to another aspect, a radiographic interpretation management method includes: first obtaining to obtain an automatically generated finding obtained by computer processing on medical information, second obtaining to obtain a radiographic interpretation finding created by a user based on the medical information, and setting to be able to set by user operation a combination that is to be displayed with highlight among combinations of a result of the automatically generated finding and a result of the radiographic interpretation finding.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a diagram showing an example of an entire configuration of a medical image system according to the present embodiment;

FIG. 2 is a block diagram of a main section showing a functional configuration of one embodiment of an analysis apparatus as a radiographic interpretation management apparatus according to the present embodiment;

FIG. 3 is a flowchart showing a flow of a radiographic interpretation management method;

FIG. 4 is a diagram showing an example of a work flow of radiographic interpretation;

FIG. 5 is a diagram showing an example of a work list display screen;

FIG. 6 is a diagram showing an example of a search condition tab in a search filter edit screen;

FIG. 7 is a diagram showing an example of a display item (examination) tab in a search filter edit screen;

FIG. 8 is a diagram showing an example of a design tab in a search filter edit screen;

FIG. 9 is a diagram showing an example of a condition setting screen;

FIG. 10 is a diagram showing an example of a condition setting screen;

FIG. 11 is a diagram showing an example of a condition setting screen;

FIG. 12 is a diagram showing an example of a work list display screen;

FIG. 13 is a diagram showing an example of a work list display screen;

FIG. 14 is a diagram showing an example of a work list display screen; and

FIG. 15 is a diagram showing an example of a work flow of radiographic interpretation.

### DETAILED DESCRIPTION

Hereinafter, embodiments of a radiographic interpretation management device, a storage medium, and a radiographic interpretation management method according to the present invention will be described. However, the scope of the present invention is not limited to the embodiments or illustrated examples.

### [Configuration of Medical Image System]

The radiographic interpretation management apparatus according to the present embodiment performs analysis of the medical image which is the medical information in the medical image system, for example. The “analysis apparatus” described below functions as the “radiographic interpretation management apparatus”.

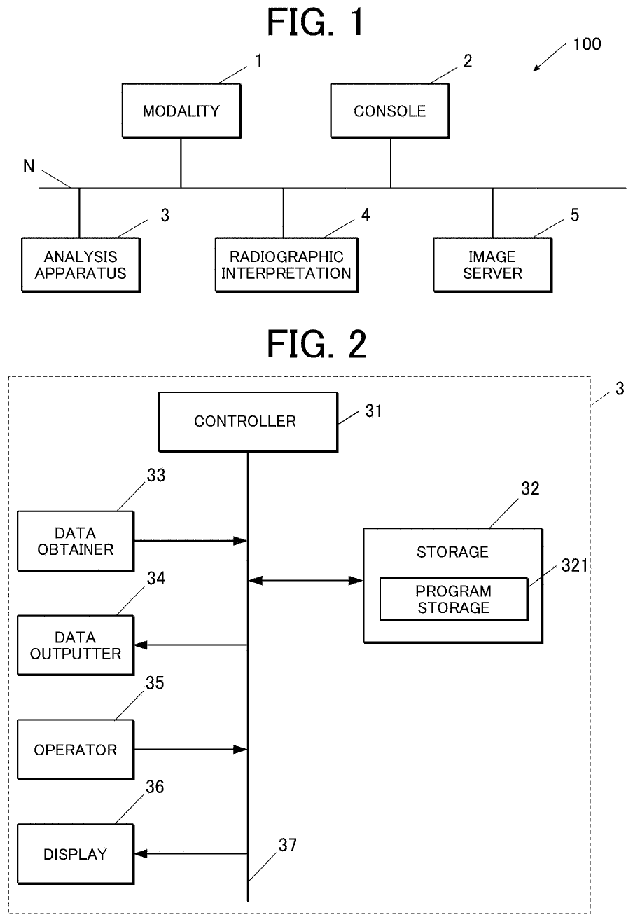

FIG. 1 is a system configuration of a medical image system 100.

As shown in FIG. 1, the medical image system 100 includes a modality 1, a console 2, an analysis apparatus 3, a radiographic interpretation terminal 4, an image server 5, and the like, and the above units are connected to each other through a communication network N such as LAN (Local Area Network), WAN (Wide Area Network), the Internet, etc. These components of the medical image system 100 are in conformity with HL7 (Health Level Seven) or DICOM (Digital Image and Communications in Medicine) standard and communicate with one another in conformity with HL7 or DICOM.

The number of units of the modality 1, the console 2, and the radiographic interpretation terminal 4 is not limited.

The modality 1 is an image generating apparatus such as a X-ray imaging apparatus (DR, CR) or an ultrasound diagnosis apparatus (US), CT, MRI, etc. Based on examination order information transmitted from an RIS (Radiology Information System, not shown), the modality 1 images an examination target site of the patient as a subject and generates a medial image as medical information. Supplementary information (patient information, examination information, image ID, etc.) is written in the medical image generated in the modality 1 in the header of the image file according to the DICOM standard. The medical information in which the supplementary information is added as described above is transmitted to the analysis apparatus 3 or the radiographic interpretation terminal 4 through the console 2.

The console 2 is an imaging control apparatus that controls imaging by the modality 1. The console 2 outputs the imaging condition and the image reading condition to the modality 1 and obtains the image data of the medical image imaged by the modality 1. The console 2 includes a controller, a display, an operator, a communicator, a storage, and the like which are not shown, and the above units are connected to each other through a bus.

The analysis apparatus 3 is an apparatus that performs various analysis on medical images which are medical information and is the radiographic interpretation management apparatus according to the present embodiment. The analysis apparatus 3 may be a PC, a portable terminal or a dedicated apparatus. The analysis apparatus 3 according to the present embodiment includes a medical image management apparatus such as PACS (Picture Archiving and Communication System).

FIG. 2 is a block diagram showing a functional configuration of an analysis apparatus 3.

As shown in FIG. 2, the analysis apparatus 3 includes a controller 31 (hardware processor), a storage 32, a data obtainer 33, a data outputter 34, an operator 35, a display 36, and the like, and the above units are connected by a bus 37.

The data obtainer 33 obtains various data from external devices (for example, console 2, radiographic interpretation terminal 4, etc.).

The data obtainer 33 includes a network interface and receives data from an external device connected wired or wirelessly to the communication network N. According to the present embodiment, the data obtainer 33 includes a network interface and may include a port in which a

USB memory or a SD card can be inserted.

According to the present embodiment, the data obtainer 33 obtains image data of the medical image from the console 2, for example. The data obtainer 33 obtains a diagnosis result regarding the medical image prepared by the user (for example, a doctor) based on the medical image which is the medical information from the radiographic interpretation terminal 4 (detected result information of a lesion which can be read from the medical image) and information such as a radiographic interpretation report which is the radiographic interpretation result by the radiographic interpretation doctor (for example, radiographic interpretation doctors that perform a first radiographic interpretation and a second radiographic interpretation).

Specifically, the data obtainer 33 functions as a second obtainer that obtains “radiographic interpretation findings” (radiographic interpretation result) prepared by the user (for example, first radiographic interpretation doctor, second radiographic interpretation doctor) based on the medical information. When the supplementary information is added such as when the user such as the radiographic interpretation doctor sets a region of interest (ROI) in the medical image, the data obtainer 33 also obtains such supplementary information.

The “radiographic interpretation findings” obtained by the data obtainer 33 includes various information such as information regarding whether there is a lesion (that is, detection of abnormal location is “+(abnormal finding present)” or “−(no abnormal finding)”), information regarding the name of the lesion and the position of the lesion location (that is lesion detected position), and the like. The specific contents of the information included in the “radiographic interpretation” is not limited to those illustrated here and may include some or may include information other than the above.

The “radiographic interpretation findings” obtained by the data obtainer 33 is sent to the controller 31.

The data outputter 34 is an outputter that outputs information processed by the analysis apparatus 3. The destination to which the data outputter 34 outputs the various information is not limited. For example, the destination may be a display 36 of an analysis apparatus 3, a radiographic interpretation terminal 4, an image server 5 or various external display apparatuses which are not shown.

As described later, according to the present embodiment, the predetermined highlight display is set based on user operation, and the information processed by the analysis apparatus 3 is output to the display 36 so that various displays are performed based on the set predetermined highlight display.

Examples of devices that can be applied as the data outputter 34 include, a network interface for communication with the radiographic interpretation terminal 4 and the image server 5, a connector to connect with the external apparatus (for example, display apparatus, printer, etc. which are not shown), a port for various media such as a USB memory, and the like.

The operator 35 includes a keyboard including various keys and a pointing device such as a mouse or a touch panel attached to the display 36. The user is able to operate the input on the operator 35, specifically, the operation signal input by key operation on the keyboard, the mouse operation or the touch operation on the touch panel is output to the controller 31.

According to the present embodiment, as described later, the user is able to customize the desired highlight display. The operator 35 receives the input operation by the user, and the input operation signal is output to the controller 31.

The display 36 includes a monitor such as an LCD (Liquid Crystal Display) and displays various screens according to instructions of display signals input from the controller 31. The number of monitors is not limited to one, and a plurality of monitors may be provided.

As described later, in the display 36, various information is suitably displayed based on the display data output from the controller 31.

As described later, the display 36 displays with highlight based on the combination in which highlight display is set in the controller 31 (setter).

As described later, the display 36 displays with highlight the combination of the result of the “automatically generated finding” and the result of the “radiographic interpretation finding” based on the combination in which the highlight display is set by the controller 31 (setter) and a threshold of a degree of confidence of the “automatically generated finding”.

The controller 31 includes a CPU (Central Processing Unit), a RAM (Random Access Memory), and the like, and the operation of each unit of the analysis apparatus 3 is centrally controlled. Specifically, the CPU reads out various processing programs stored in the program storage 321 of the storage 32 and deploys the programs in the RAM. The various processing is performed according to the program. In coordination with the program, the controller 31 according to the present embodiment realizes various functions as described below.

For example, the controller 31 functions as a “first obtainer” that obtains an “automatically generated finding” obtained by the computer processes on the medical information.

Specifically, detecting and analysis processing of the lesion is performed on the medical image obtained by the data obtainer 33, and a detection/analysis result of one or a plurality of types of lesions is output.

Here, as the computer processing, AI analysis using AI (Artificial Intelligence) that performs image diagnosis/image analysis including detection of the lesion by CAD (Computer Aided Diagnosis) for example, is used.

According to the present embodiment, the controller 31 as the “first obtainer” obtains the result of whether the lesion is detected (that is, the result of the detection of the abnormal location is “+(there is abnormal finding)” or “−(there is no abnormal finding)”) and the result of the detected position of the lesion location (that is, position where the lesion is detected) as the “automatically generated finding”. However, the “automatically generated finding” is not limited to the above. For example, the controller 31 may function as a learner (not shown) that learns the correspondence between the medical information (medical image of the present embodiment) and information related to the field of medicine (name of lesion, etc.) and the “automatically generated finding” may be obtained by computer processing of the medical information (medical image) based on the correspondence between the learned medical information (medical image) and the information related to the field of medicine.

That is, the detection/analysis process of the lesion is performed on the input medical image based on a machine learning model created by training by deep learning using a large amount of learning data (a medical image showing the lesion paired with a correct label (diagnosis name (type of lesion) of the lesion in the medical image, etc.).

When the “automatically generated finding” is obtained as described above, information related to the name of the lesion, for example is added to the image data of the medical image as the supplementary information.

According to the present embodiment, when radiographic interpretation of the medical information (medical image according to the present embodiment) is performed, the controller 31 contrasts the “automatically generated finding” which is the analysis result by the AI with the “radiographic interpretation finding” by the radiographic interpretation doctor. With this, it is possible to achieve quality assurance (QA) regarding the diagnosis accuracy of the medical information (medical image).

For example, when information showing that an abnormal location is detected (that is, “+” showing that there is an abnormal finding) is extracted from the “automatically generated finding” and information showing that an abnormal location is detected (that is, “+” showing that there is an abnormal finding) is also extracted from the “radiographic interpretation finding”, the “automatically generated finding” by the AI and the radiographic interpretation finding by the doctor both become “+” (there is abnormal finding). The controller 31 derives (calculates) the comparison result that there is a match between both results showing “+” (there is abnormal finding).

On the other hand, when information showing that an abnormal location is detected (that is, “+” showing that there is an abnormal finding) is extracted from the “automatically generated finding” and information showing that an abnormal location is not detected (that is, “−” showing that there is no abnormal finding) is extracted from the “radiographic interpretation finding”, the controller 31 derives (calculates) the comparison result showing that there is no match (there is a difference) between the “automatically generated finding” by the AI analysis showing “+” (there is abnormal finding) and the radiographic interpretation finding by the doctor showing “−” (there is no abnormal finding).

For example, when information showing that an abnormal location is detected and the position of the lesion location is “XX” is extracted from the “automatically generated finding” and information showing that an abnormal location is detected and the position of the lesion location is “XX” is extracted from the “radiographic interpretation finding” also, the “automatically generated finding” by the AI and the radiographic interpretation finding by the doctor both become information showing an abnormal finding is detected and the position of the lesion location is “XX”. The controller 31 derives (calculates) the comparison result that there is a match in both results.

On the other hand, when information showing that an abnormal location is detected and the position of the lesion location is “XX” is extracted from the “automatically generated finding” but information showing that an abnormal location is detected and the position of the lesion location is “YY” is extracted from the “radiographic interpretation finding”, the controller 31 derives (calculates) the comparison result that there is no match (there is a difference) in the position of the lesion location as a result of the comparison between the “automatically generated finding” by the AI analysis and the “radiographic interpretation finding” by the doctor.

According to the present embodiment, the information extracted from the “automatically generated finding” may be information showing “+” representing that there is an abnormal finding and information showing “−” representing that there is no abnormal finding. When there is an abnormal finding, the information of the position where the lesion is detected may be extracted.

The information extracted from the “radiographic interpretation finding” may also be information showing “+” representing that there is an abnormal finding and information showing “−” representing that there is no abnormal finding. When there is an abnormal finding, the information of the position where the lesion is detected may be extracted.

The information extracted from the “automatically generated finding” and the “radiographic interpretation finding” is not limited to whether there is an abnormal finding and the position where the lesion is detected and may also be type or number of abnormal findings. According to the embodiment described below, the controller 31 performs comparison (contrast) processing regarding whether there is an abnormal finding and the position where the lesion is detected.

The “automatically generated finding” is structured data obtained by computer processing (AI analysis). On the other hand, the “radiographic interpretation finding” may be a radiographic interpretation report prepared by the doctor, and is not limited to structured data.

For example, whether there is an abnormal finding may be represented by “+” or “−” or may be represented by a string of letters showing whether there is the abnormal finding such as the abnormality is “present” or “not present” or the abnormality “can be confirmed” or “cannot be confirmed”. That is, there may be a variation in the expression. Therefore, the controller 31 may structure the “radiographic interpretation finding” as data (structured data) including a string of letters that can be contrasted (compared) with the “automatically generated finding” as a precondition for contrast between the “automatically generated finding” and the “radiographic interpretation finding”. In this case, for example, the storage 32 may store dictionary data defining the correspondence of the string of letters used to generate the structure data. The controller 31 refers to the above and structures the “radiographic interpretation finding”.

When the controller 31 is able to define the various expressions and terms in the “radiographic interpretation finding” widely, even if there are some differences in the expressions between the “automatically generated finding” and the “radiographic interpretation finding”, it is possible to determine whether there is a match or not. Examples of the above may be when it is possible to correspond expressions such as “+”, abnormality is “present”, and abnormality “can be confirmed” as meaning the same as “information showing that there is an abnormal finding (+)” extracted from the “automatically generated finding”. Other examples of the above may be when it is possible to correspond expressions such as “−”, abnormality is “not present” and abnormality “cannot be confirmed” as meaning the same as “information showing that there is no abnormality finding (−)” extracted from the “automatically generated finding”. In this case, the contrast (comparison) processing between the “automatically generated finding” and the “radiographic interpretation finding” can be performed without structuring the “radiographic interpretation finding”.

The controller 31 according to the present embodiment functions as a “setter” that is able to set by the user operation a combination that is to be displayed with highlight among the combinations of the result of the “automatically generated finding” and the “radiographic interpretation finding”.

According to the present embodiment, the controller 31 is able to set the predetermined highlight display.

For example, according to user operation, the controller 31 as the setter is able to set to display with highlight the combination showing that the result of the “automatically generated finding” is the lesion is detected (detection of the abnormal location is “+”), and the result of the “radiographic interpretation finding” is the lesion is not detected (detection of the abnormal location is “−”) among the combinations of the result of the “automatically generated finding” and the result of the “radiographic interpretation finding”.

Alternatively, according to user operation, the controller 31 as the setter is able to set to display with highlight the combination showing that the result of the “automatically generated finding” is the lesion is not detected (detection of the abnormal location is “−”), and the result of the “radiographic interpretation finding” is the lesion is detected (detection of the abnormal location is “+”) among the combinations of the result of the “automatically generated finding” and the result of the “radiographic interpretation finding”.

Alternatively, according to user operation, the controller 31 as the setter is able to set to display with highlight the combination showing that the result of the “automatically generated finding” is the lesion is not detected (detection of the abnormal location is “−”), and the result of the “radiographic interpretation finding” is the lesion is not detected (detection of the abnormal location is “−”) among the combinations of the result of the “automatically generated finding” and the result of the “radiographic interpretation finding”.

Alternatively, according to user operation, the controller 31 as the setter is able to set to display with highlight the combination showing that the result of the “automatically generated finding” is the lesion is detected (detection of the abnormal location is “+”), and the result of the “radiographic interpretation finding” is the lesion is detected (detection of the abnormal location is “+”) among the combinations of the result of the “automatically generated finding” and the result of the “radiographic interpretation finding”.

For example, according to user operation, the controller 31 as the setter is able to set to display with highlight the combination including a first region that is detected as the position where the lesion is detected in the “automatically generated finding” and a second region that is different from the first region and that is detected as the position where the lesion is detected in the “radiographic interpretation finding” among the combinations of the result of the detected position of the lesion in the “automatically generated finding” and the result of the detected position of the lesion in the “radiographic interpretation finding”.

For example, according to user operation, the controller 31 as the setter is able to set the combination that is to be displayed with highlight among the combinations of the result of the “automatically generated finding” and the result of the “radiographic interpretation finding” and the threshold of the degree of confidence of the “automatically generated finding”.

For example, according to the user operation, the combination including the result of the “automatically generated finding” showing that there is an abnormal finding and the result of the “radiographic interpretation finding” showing that there is no abnormal finding is set as the combination to be displayed with highlight. In this case, regarding the combination, the threshold of the degree of confidence of the “automatically generated finding” is set as the threshold of the highlight display.

Here, when the degree of confidence of the “automatically generated finding” is equal to or larger than the set threshold, the combination of the result of the “automatically generated finding” and the result of the “radiographic interpretation finding” set as the combination to be displayed with highlight is displayed with highlight. On the other hand, when the degree of confidence of the “automatically generated finding” is equal to or smaller than the set threshold, the combination of the result of the “automatically generated finding” and the result of the “radiographic interpretation finding” set as the combination to be displayed with highlight is not displayed with highlight.

The specific highlight display setting process is described later.

The controller 31 according to the present embodiment functions as the “display controller” that displays with highlight based on the combination that is to be displayed with highlight set by the setter.

The controller 31 as the display controller displays with highlight the combination of the result of the “automatically generated finding” and the result of the “radiographic interpretation finding” based on the combination that is to be displayed with highlight set by the setter and the threshold of the degree of confidence of the “automatically generated finding” set by the setter.

The storage 32 includes a HDD (Hard Disk Drive) and a semiconductor memory, and includes a program storage 321 that stores the program to execute various processes such as the setting process of the highlight display described later. The parameters, files, etc. necessary to execute the program stored in the program storage 321 are stored in the storage 32.

As described above, when the controller 31 performs the process to structure the unstructured data such as the radiographic interpretation report prepared by the user (radiographic interpretation doctor) and to generate the structured data that can be contrasted with the “automatically generated finding”, dictionary data (structuring dictionary) used to perform a structuring process is stored in the storage 32.

The radiographic interpretation terminal 4 is a computer apparatus that includes, for example, a controller, an operator, a display, a storage, a communicator, and the like. The radiographic interpretation terminal 4 reads the medical image which is the medical information from the image server 5 and displays the medical image for radiographic interpretation.

The user (first radiographic interpretation doctor, second radiographic interpretation doctor, etc.) performs radiographic interpretation of the medical image with the radiographic interpretation terminal 4 and prepares the radiographic interpretation report that is the diagnosis result of the radiographic interpretation doctor regarding the medical image.

For example, the image server 5 is a server included in PACS (Picture Archiving and Communication System) and stores the following items corresponded to the medical image output from the modality 1 in a database, examples of the items including patient information (patient ID, patient name, birthday, age, sex, height, weight, etc.), examination information (examination ID, examination date/time, type of modality, examination site, requesting department, examination purpose, etc.), image ID of medical image, information of analysis result of AI output from the controller 31 of the analysis apparatus 3 (that is, automatically generated finding) and radiographic interpretation finding by the radiographic interpretation doctor such as radiographic interpretation report prepared by the user (radiographic interpretation doctor) on the radiographic interpretation terminal 4 (that is, radiographic interpretation finding, etc.) and result of contrast (comparison result) output by the controller 31 of the analysis apparatus 3.

[Radiographic Interpretation Management Method according to Present Embodiment]

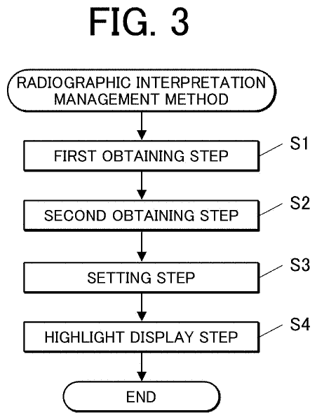

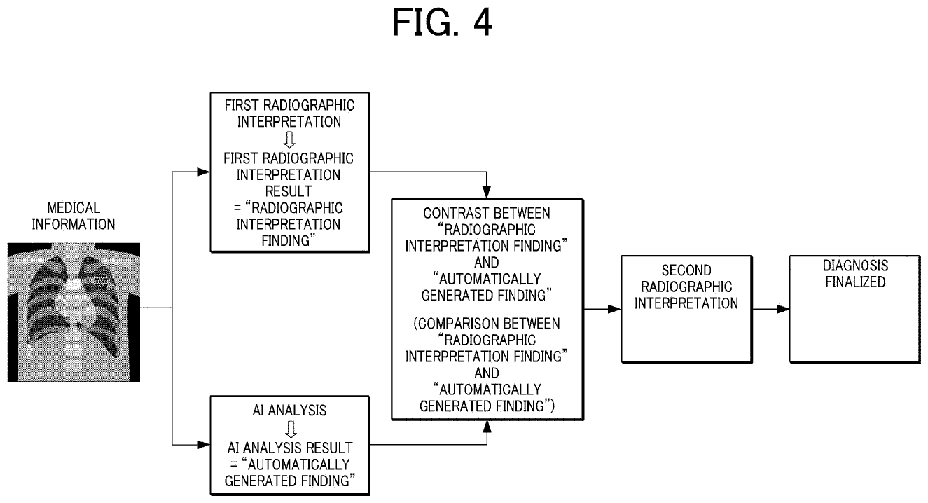

The radiographic interpretation management method according to the present embodiment includes the following steps as shown in FIG. 3. In the first obtaining step, the “automatically generated finding” obtained by computer processing (AI analysis according to the present embodiment) on the medical information is obtained (step S1). In the second obtaining step, the “radiographic interpretation finding” prepared by the user is obtained based on the medical information (step S2). In the setting step, the user is able to set by user operation the combination that is to be displayed with highlight among the combinations of the result of the “automatically generated finding” and the result of the “radiographic interpretation finding” (step S3). In the highlight display step, the highlight display set in the setting step is performed (step S4).

The setting step S3 may be performed before the first obtaining step Si and the second obtaining step S2.

FIG. 4 shows an example of a work flow in the radiographic interpretation. In the example shown in FIG. 4, for example, first radiographic interpretation by the doctor (first radiographic interpretation doctor) is performed on the medical information and the AI analysis by the controller 31 is performed. Then, the “automatically generated finding” which is the analysis result by the AI is contrasted with the “radiographic interpretation finding” which is the radiographic interpretation result of the first radiographic interpretation doctor. Then, the result is sent to the second radiographic interpretation doctor (second radiographic interpretation reader) to perform the second radiographic interpretation. The result of the contrast between the “automatically generated finding” and the “radiographic interpretation finding” which is the radiographic interpretation result by the first radiographic interpretation doctor is displayed on the work list of the second radiographic interpretation doctor (second radiographic interpretation reader). Then, the second radiographic interpretation doctor performs the second radiographic interpretation by referring to the contrast result displayed on the work list and the radiographic interpretation report by the first radiographic interpretation doctor which is the “radiographic interpretation finding”. Then, the diagnosis is finalized.

### (Work List Display Setting Process)

The above-mentioned display setting process of the work list (for the second radiographic interpretation doctor (second radiographic interpretation reader), for example) is described below. According to the present embodiment, a list of examination information (examination list) including the result of the “automatically generated finding” (AI determination result) and the result of the “radiographic interpretation finding” (user determination result) is displayed on the work list.

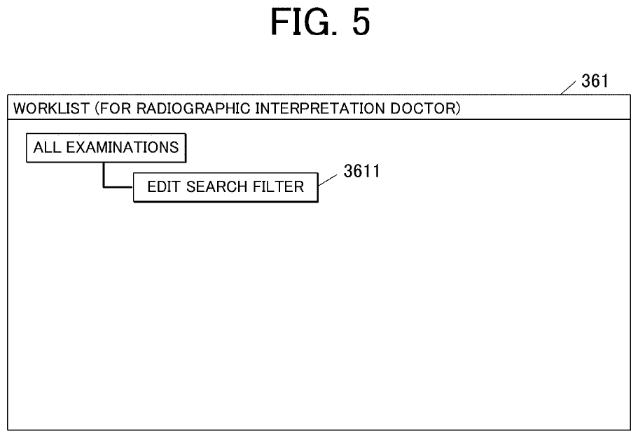

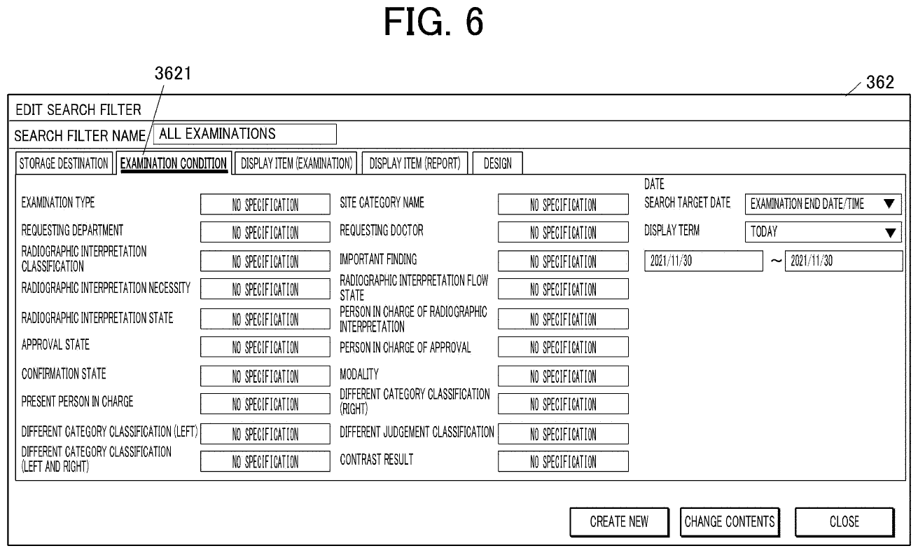

First, when search filter edit 3611 is selected by input operation by the user (for example, second radiographic interpretation doctor) through the operator 35 on a work list display screen 361 displayed on the display 36 as shown in FIG. 5, the controller 31 controls the display 36 to display a search filter edit screen 362 as shown in FIG. 6.

According to the present embodiment, the user is able to specify the desired search condition in the examination information displayed on the work list display screen 361.

The controller 31 accepts specification of the search condition of the examination information displayed on the work list display screen 361 in a search condition tab 3621 on the search filter edit screen 362.

According to the example shown in FIG. 6, examples of the search condition that can be specified include, examination type, site category name, requesting department, requesting doctor, radiographic interpretation classification, important finding, radiographic interpretation necessity, radiographic interpretation flow state, radiographic interpretation state, person in charge of radiographic interpretation, approval state, person in charge of approval, confirmation state, modality, present person in charge, different category classification (right), different category classification (left), different judgement classification, different category classification (left and right), contrast result, and examination date. The search condition is not limited to the above, and other items may be the search condition.

For example, when “X-ray simple imaging” is specified as the examination type in the search condition, the controller 31 extracts the examination information in which the examination type is the “X-ray simple imaging” among the examination information stored in the image server 5. Then, the controller 31 displays the extracted search information on the work list display screen 361.

When “radiographic interpretation doctor A” in the person in charge of the radiographic interpretation is specified as the search condition, the controller 31 extracts the examination information in which the person in charge of the radiographic interpretation is the “radiographic interpretation doctor A” among the examination information stored in the image server 5. Then, the controller 31 displays the extracted search information on the work list display screen 361.

According to the present embodiment, the item of the examination information displayed on the work list display screen 361 can be specified by the user.

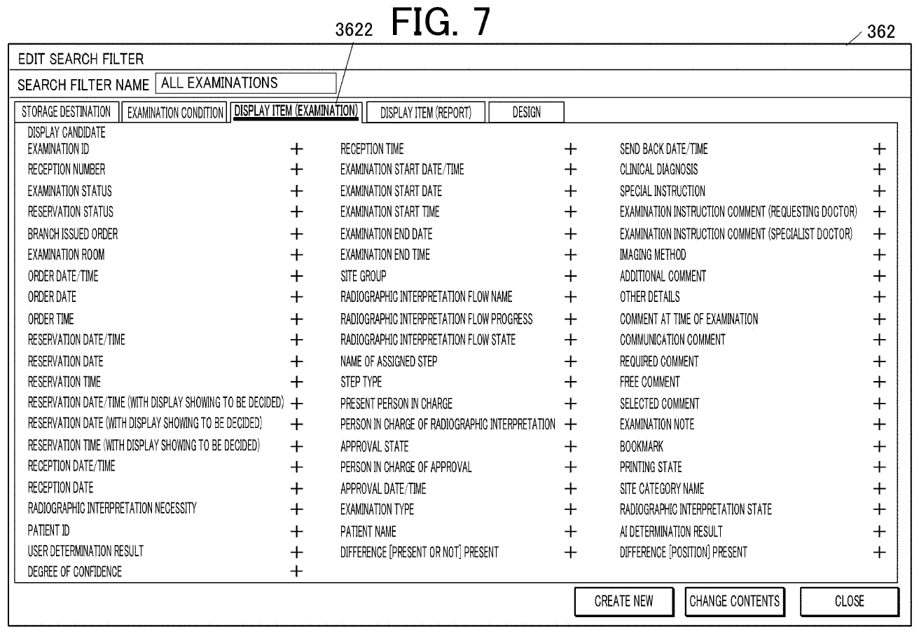

The controller 31 accepts specification of the item of the examination information displayed on the work list display screen 361 in a display item (examination) tab 3622 on the search filter edit screen 362 shown in FIG. 7.

According to the example shown in FIG. 7, the items of the examination information to be displayed can be specified. Examples of the items of the examination information to be displayed include examination ID, reception number, examination status, reservation status, branch issued order, examination room, order date/time, order date, order time, reservation date/time, reservation date, reservation time, reservation date/time (with display showing to be decided), reservation date (with display showing to be decided), reservation time (with display showing to be decided), reception date/time, reception date, reception time, examination start date/time, examination start date, examination start time, examination end date, examination end time, site group, radiographic interpretation flow name, radiographic interpretation flow progress, radiographic interpretation flow state, name of assigned step, step type, present person in charge, person in charge of radiographic interpretation, approval state, person in charge of approval, approval date/time, send back date/time, clinical diagnosis, special instruction, examination instruction comment (requesting doctor), examination instruction comment (specialist doctor), imaging method, additional comment, other details, comment at the time of examination, communication comment, required comment, free comment, selected comment, examination note, bookmark, printing state, site category name, radiographic interpretation necessity, examination type, radiographic interpretation state, patient ID, patient name, AI determination result, user determination result, difference [present or not] present, difference [position] present, and degree of confidence (of “automatically generated finding”). The items of the examination information to be displayed are not limited to the above, and other items may be specified as the items of the examination information to be displayed.

The controller 31 performs control to display the specified items of the examination information on the work list display screen 361.

The difference [present or not] shows the difference regarding whether the lesion exists in the contrast result (comparison result) between the “automatically generated finding” and the “radiographic interpretation finding”.

Specifically, when the “automatically generated finding” shows “no lesion” (that is, “no abnormality (−)”) and the “radiographic interpretation finding” also shows “no lesion” (that is, “no abnormality (−)”), the result of contrast of the above (comparison result) is “match” (that is, no difference).

When the “automatically generated finding” shows “lesion is present” (that is, “abnormality is present (+)”) and the “radiographic interpretation finding” also shows “lesion is present” (that is, abnormality is present (+)”), the result of contrast of the above (comparison result) is “match” (that is, no difference).

When the “automatically generated finding” shows “lesion is present” (that is, “abnormality is present (+)”) and the “radiographic interpretation finding” shows “no lesion” (that is, “no abnormality (−)”), the result of contrast of the above (comparison result) is “no match” (that is, difference is present).

When the “automatically generated finding” shows “no lesion” (that is, “no abnormality (−)”) and the “radiographic interpretation finding” shows “lesion is present” (that is, “abnormality is present (+)”), the result of contrast of the above (comparison result) is “no match” (that is, difference is present).

The difference [position] shows the difference of the position of where the lesion is detected in the contrast result (comparison result) between the “automatically generated finding” and the “radiographic interpretation finding”.

Specifically, when the “automatically generated finding” shows “lesion position: XX” and the “radiographic interpretation finding” also shows “lesion position: XX”, the result of contrast of the above (comparison result) is “match” (that is, no difference).

Specifically, when the “automatically generated finding” shows “lesion position: XX” and the “radiographic interpretation finding” shows “lesion position: YY”, the result of contrast of the above (comparison result) is “no match” (that is, difference is present).

### (Highlight Display Setting Process)

According to the present embodiment, the user is able to set the desired highlight display in the examination information displayed on the work list display screen 361.

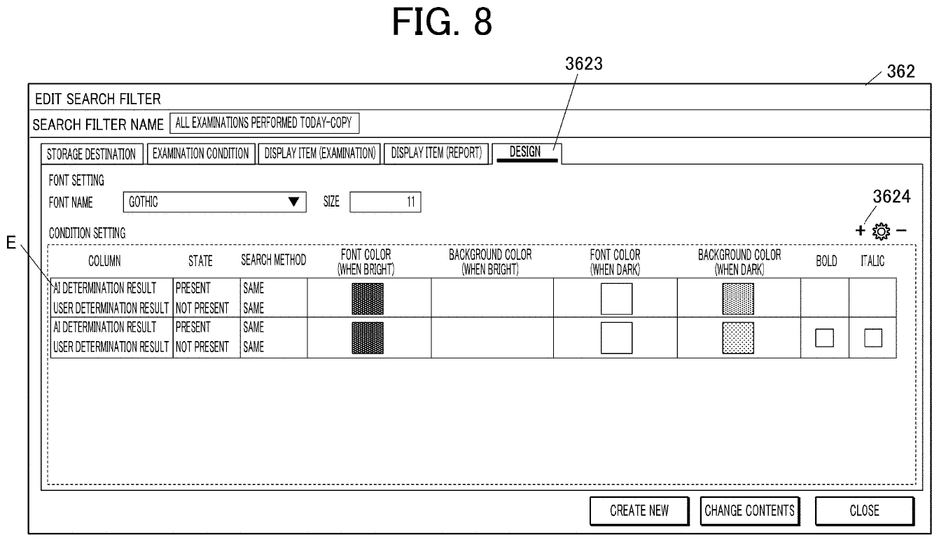

The controller 31 accepts setting of the highlight display of the examination information displayed on the work list display screen 361 in a design tab 3623 on the search filter edit screen 362 shown in FIG. 8. This process is the highlight display setting process.

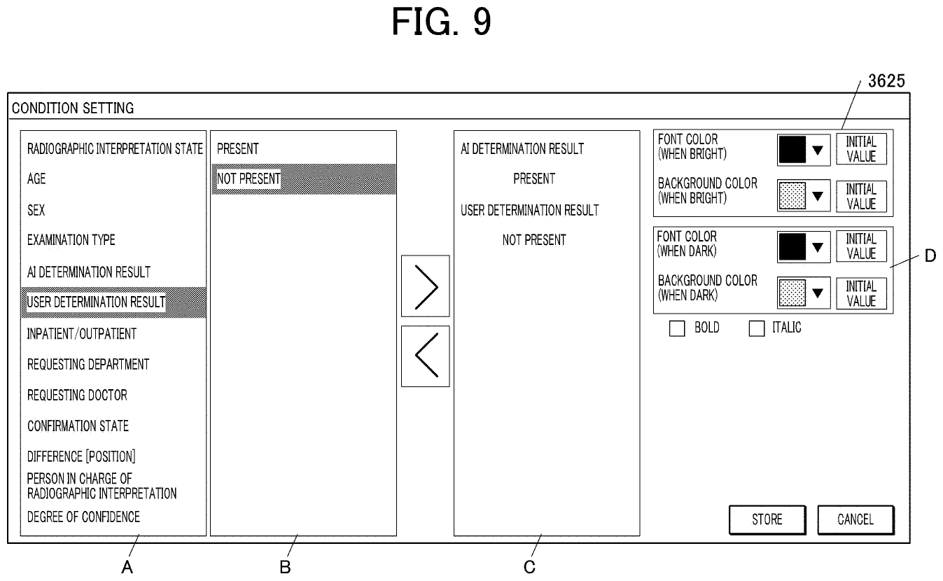

In the design tab 3623 shown in FIG. 8, when a condition setting button 3624 is pressed by input operation by the user through the operator 35, the controller 31 controls the display 36 to display a condition setting screen 3625 shown in FIG. 9.

The controller 31 accepts setting of the condition of the highlight display of the examination information displayed on the work list display screen 361 on the condition setting screen 3625.

Specifically, the controller 31 receives the conditions of the highlight display in condition item A and condition state B shown in FIG. 9.

First, the controller 31 accepts the condition item that is displayed with highlight in the condition item A. According to the example shown in FIG. 9, the condition items that can be specified to be displayed with highlight include, radiographic interpretation state, age, sex, examination type, AI determination result (result of “automatically generated finding”), user determination result (result of “radiographic interpretation finding”), inpatient/outpatient (inpatient, outpatient, or outpatient during hospitalization), requesting department, requesting doctor, confirmation state, difference [position], person in charge of radiographic interpretation (radiographic interpretation doctor that performs radiographic interpretation), and degree of confidence (degree of confidence of “automatically generated finding”). The condition item displayed with highlight is not limited to the above and other items may be specified as the condition item displayed with highlight.

Then, the controller 31 accepts the state of the condition that is displayed with highlight in the condition state B.

For example, according to the example shown in FIG. 9, “user determination result” is specified in the condition item A. In this case, in the condition state B, it is possible to specify whether the “user determination result” shows “(lesion) present” or “(lesion) not present”.

Then, the controller 31 performs control to display the conditions of the highlight display specified in the condition item A and the condition state B in condition display C shown in FIG. 9.

According to the example shown in FIG. 9, as the condition to be displayed with highlight, “AI determination result: (lesion) present” and “user determination result: (lesion) not present” are specified.

Similarly, as the condition to be displayed with highlight, “AI determination result: (lesion) not present” and “user determination result: (lesion) present” can be specified.

Similarly, as the condition to be displayed with highlight, “AI determination result: (lesion) not present” and “user determination result: (lesion) not present” can be specified.

Similarly, as the condition to be displayed with highlight, “AI determination result: (lesion) present” and “user determination result: (lesion) present” can be specified.

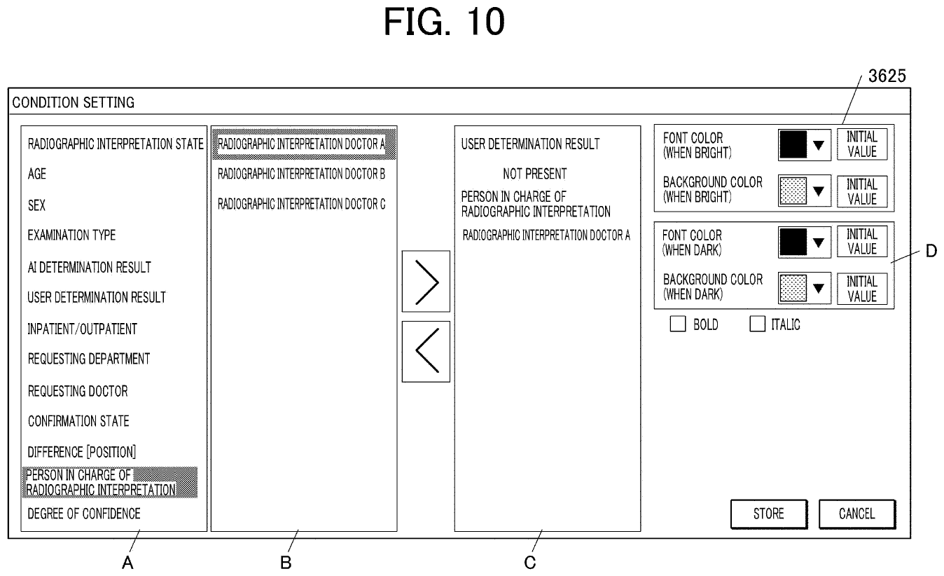

According to the example shown in FIG. 10, “person in charge of radiographic interpretation” is specified in the condition item A. In this case, in the condition state B, the “person in charge of radiographic interpretation” can be specified from “radiographic interpretation doctor A”, “radiographic interpretation doctor B”, or “radiographic interpretation doctor C”.

According to the example shown in FIG. 10, the “user determination result: (lesion) not present” and “person in charge of radiographic interpretation: radiographic interpretation doctor A” are specified as the conditions to be displayed in highlight in the condition display C.

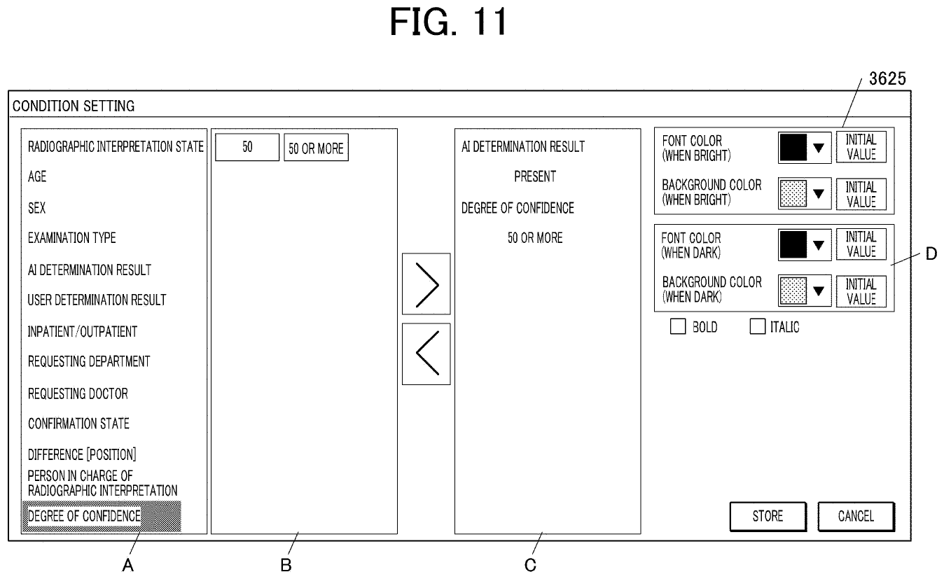

According to the example shown in FIG. 11, the “degree of confidence” (degree of confidence of “automatically generated finding”) is specified in the condition item A. In this case, a threshold of the “degree of confidence” can be specified in the condition state B. According to the example shown in FIG. 11, the “degree of confidence” is specified to be “50 or more” but the user is able to freely set the value of the threshold and whether equal to or more or equal to or less than the threshold through the operator 35.

According to the example shown in FIG. 11, “AI determination result: (lesion) present” and “degree of confidence: 50 or more” are specified as the condition to display with highlight in the condition display C.

Then, the controller 31 accepts the specification of the manner of the highlight display in display manner D shown in FIG. 9 to FIG. 11.

For example, according to the example shown in FIG. 9 to FIG. 11, font color, background color, bold text, and italic text can be specified as the manner of the highlight display. The manner of the highlight display is not limited to the above and other manner of display may be employed. Any manner of display may be employed as long as the difference from the display of the examination information which is not highlighted can be recognized. The manner of display in which the difference can be recognized includes displaying in a manner different from the examination information which is not displayed with highlight. The highlight display includes displaying a predetermined combination of the result of the automatically generated finding and the result of the radiographic interpretation finding in a manner so that the difference from the display of other combinations can be recognized. The display in which the difference can be recognized includes displaying in a manner different from the display of other combinations, for example.

Specifically, in addition to the above, the manner of highlight displays includes font of the text, blinking of the text, and the like. As an item of the examination information displayed on the work list display screen 361, an item may be added for displaying a mark showing that the highlight display is set, and the mark showing the highlight display may be added in the space of the item provided for the examination information in which highlight display is set. Alternatively, the order of display and the position of the examination information in which highlight display is set can be changed. Specifically, for example, the examination information in which the highlight display is set can be displayed at the top of the work list.

As described above, the manner of the highlight display includes change and blinking in the color and the text. Specifically, the change in the color includes the change in the font color and the change in the background color. The change of the text includes the change to bold text, the change to italic text, and the change of the font of the text. Blinking includes the text blinking and the color blinking. The display with highlight includes adding an item for highlight display and adding a mark in such item. The display with highlight includes displaying so that a predetermined row or column stand out or changing the order of display, the position of display, and the priority of display of the predetermined row or column.

As described above, the condition of the highlight display set in the condition setting screen 3625 is displayed in the column E in the design tab 3623 shown in FIG. 8.

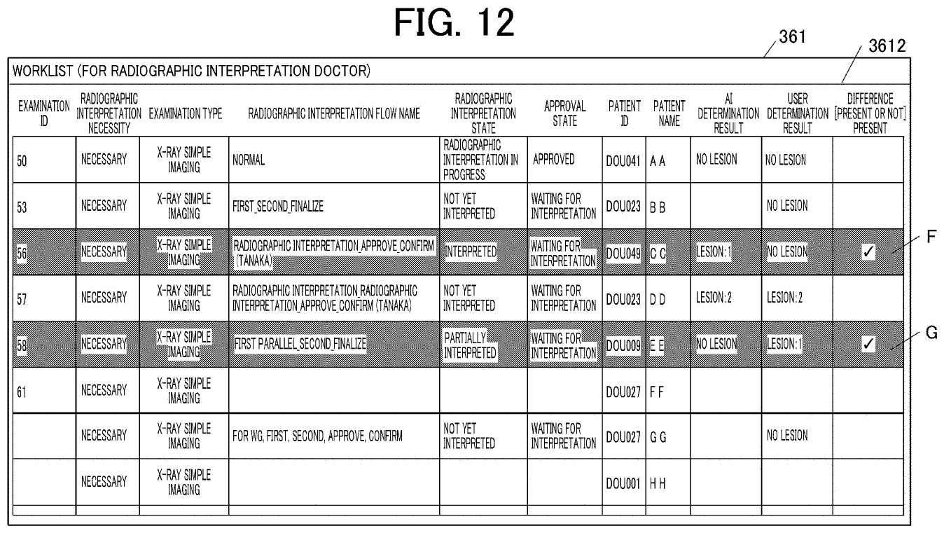

Next, FIG. 12 shows an example of a work list 3612 displayed on the work list display screen 361.

According to the example shown in FIG. 12, the examination information displayed on the work list 3612 is the examination information when the examination information in which “X-ray simple imaging” is specified as the examination type in the search condition tab 3621. That is, the controller 31 extracts the examination information in which “X-ray simple imaging” is specified as the examination type from the examination information stored in the image server 5, and the information is displayed in the work list 3612.

As the example shown in FIG. 12, the item of the examination information displayed in the work list 3612 includes the following items specified in the display item (examination) tab 3622, for example, examination ID, radiographic interpretation necessity, examination type, radiographic interpretation flow name, radiographic interpretation state, approval state, patient ID, patient name, AI determination result, user determination result, and difference [present or not] present.

According to the example shown in FIG. 12, examination information F and examination information G are displayed with the background color different from the other examination information and display with highlight is performed.

The examination information F shows that the AI determination result is “lesion: 1 (lesion present)” and the user determination result is “no lesion”.

That is, when the condition to highlight display is specified in the condition setting screen 3625 with the conditions “AI determination result: (lesion) present” and the “user determination result: (lesion) none”, the controller 31 displays with highlight the examination information that satisfy the specified conditions as shown in the examination information F.

The examination information G shows that the AI determination result is “no lesion” and the user determination result is “lesion: 1 (lesion present)”.

That is, when the condition to highlight display is specified in the condition setting screen 3625 with the conditions “AI determination result: (lesion) none” and the “user determination result: (lesion) present”, the controller 31 displays with highlight the examination information that satisfy the specified conditions as shown in the examination information G. According to the example shown in FIG. 12, when there is a difference as the comparison result between the AI determination result and the user determination result, a “checkmark” is displayed in the space of the difference [present or not] present.

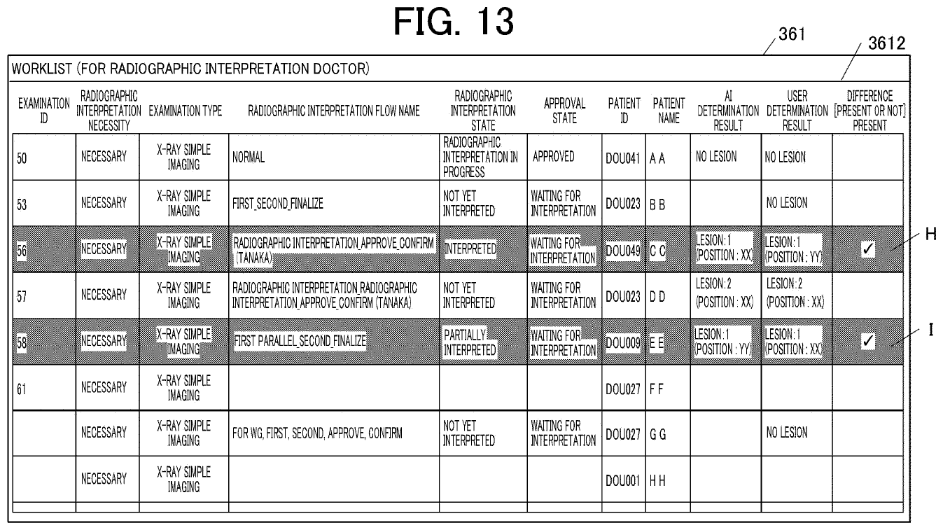

FIG. 13 shows an example of the work list 3612 in which the highlight display is set differently from FIG. 12. The portions different from the work list 3612 shown in FIG. 12 are mainly described.

According to the example shown in FIG. 13, difference [position] present is specified as the item of the examination information displayed in the work list 3612.

According to the example shown in FIG. 13, the examination information H and the examination information I are displayed with the background color different from the other examination information and display with highlight is performed.

The examination information H shows the AI determination result is “lesion: 1 (lesion present)”/(detected position of lesion: XX)” and the user determination result is “lesion: 1 (lesion present)/(detected position of lesion: YY)”, and “difference [position]: present”.

The examination information I shows the AI determination result is “lesion: 1 (lesion present)”/(detected position of lesion: YY)” and the user determination result is “lesion: 1 (lesion present)/(detected position of lesion: XX)”, and “difference [position]: present”.

That is, when the condition to highlight display is specified in the condition setting screen 3625 with the condition “difference [position]: present”, the controller 31 displays with highlight the examination information that satisfy the specified conditions as shown in the examination information H and the examination information I.

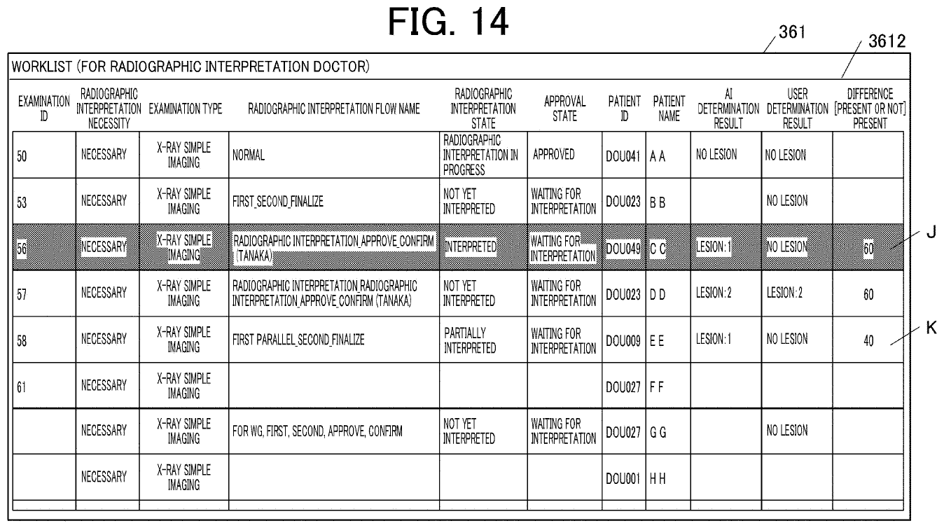

FIG. 14 shows an example of the work list 3612 in which the highlight display is set differently from FIG. 12 and FIG. 13. The portions different from the work list 3612 shown in FIG. 12 are mainly described.

According to the example shown in FIG. 14, the degree of confidence is specified as the item of the examination information displayed in the work list 3612.

According to the example shown in FIG. 14, the examination information J is displayed with the background color different from the other examination information and display with highlight is performed.

The examination information J shows that the AI determination result is “lesion: 1 (lesion present)” and the user determination result is “no lesion”, and the degree of confidence is “60”.

That is, when the condition to highlight display is specified in the condition setting screen 3625 with the conditions “AI determination result: (lesion) present” and the “user determination result: (lesion) none” and the “degree of confidence: 50 or more”, the controller 31 displays with highlight the examination information that satisfy the specified conditions as shown in the examination information J.

According to the example shown in FIG. 14, when the condition to highlight display is specified in the condition setting screen 3625 with the conditions “AI determination result: (lesion) present” and the “user determination result: (lesion) none” and the “degree of confidence: 50 or less”, the controller 31 displays with highlight the examination information that satisfies the specified conditions, for example, the examination information K.

### [Effects]

As described above, the analysis apparatus 3 as the radiographic interpretation management apparatus according to the present embodiment includes a first obtainer (controller 31) that obtains the “automatically generated finding” obtained by a computer process on the medical information, a second obtainer (data obtainer 33) that obtains the “radiographic interpretation finding” prepared by the user based on the medical information, and a setter (controller 31) in which the combination which is to be displayed with highlight among the combinations of the result of the “automatically generated finding” and the result of the “radiographic interpretation finding” can be set by user operation.

Therefore, for example, when the highlight display desired by the medical facility is different when the radiographic interpretation is performed, a specific highlight display can be set according to the needs of the user. Therefore, it is possible to achieve setting of a more favorable highlight display according to the environment of the facility, the operating conditions of the facility, the difference in experience and preference among the users (doctor, radiographic interpretation doctor), and the like.

According to the present embodiment, in the controller 31 as the setter, the user is able to set by user operation which combination to display with highlight among the combinations of the result of the “automatically generated finding” and the result of the “radiographic interpretation finding”.

Therefore, when the user (for example, second radiographic interpretation doctor) desires to confirm a certain combination of the result of the “automatically generated finding” and the result of the “radiographic interpretation finding”, the display with highlight can be performed.

According to the present embodiment, the result of the “automatically generated finding” includes the result of whether the lesion is detected by the “automatically generated finding” and the result of the “radiographic interpretation finding” includes the result of whether the lesion is detected by the “radiographic interpretation finding”.

Therefore, when the user desires to confirm a certain combination of the result of whether the lesion is detected in the “automatically generated finding” and the result of whether the lesion is detected in the “radiographic interpretation finding”, the combination can be displayed with highlight.

According to the present embodiment, the result of the “automatically generated finding” includes the result of the detected position of the lesion detected by the “automatically generated finding” and the result of the “radiographic interpretation finding” includes the result of the detected position of the lesion detected by the “radiographic interpretation finding”.

Therefore, when the user desires to confirm a certain combination of the result of the detected position of the lesion detected by the “automatically generated finding” and the result of the detected position of the lesion detected by the “radiographic interpretation finding”, the display with highlight can be performed. That is, for example, when the result of the “automatically generated finding” and the result of the “radiographic interpretation finding” both show that there is a lesion but the position of the detected lesion is different, the display can be shown with highlight.

The analysis apparatus 3 according to the present embodiment includes the display 36 which displays with highlight based on the combination in which highlight display is set in the setter.

Therefore, when the combination of the result of the “automatically generated finding” and the result of the “radiographic interpretation finding” applies to the combination in which highlight display is set by the setter, the combination can be displayed with highlight and shown to the user.

According to the present embodiment, by using the controller 31 as the setter, it is possible to set by user operation the combination to display with highlight among the combinations of the result of the “automatically generated finding” and the result of the “radiographic interpretation finding” and the threshold of the degree of confidence of the “automatically generated finding”.

The present embodiment further includes a display 36 in order to display with highlight the combination of the result of the “automatically generated finding” and the result of the “radiographic interpretation finding” based on the combination in which highlight display is set by the setter and the threshold of the degree of confidence of the “automatically generated finding” set by the setter.

Therefore, when the user desires highlight display taking into consideration the degree of confidence of the “automatically generated finding”, it is possible to present to the user displayed with highlight the combination of the result of the “automatically generated finding” and the result of the “radiographic interpretation” that is the combination that the user desires to display with highlight and that includes the degree of confidence desired by the user.

According to the present embodiment, the display 36 displays with highlight the combination of the result of the “automatically generated finding” and the result of the “radiographic interpretation finding” when the degree of confidence of the “automatically generated finding” is equal to or larger than the threshold.

Therefore, when the user desires to confirm only the result of the “automatically generated finding” with a high degree of confidence (equal to or larger than the threshold) the combination of the result of the “automatically generated finding” and the result of the “radiographic interpretation finding” including the “automatically generated finding” with the high degree of confidence can be displayed with highlight.

According to the present embodiment, when the degree of confidence of the “automatically generated finding” is equal to or smaller than the threshold, the display 36 displays with highlight the combination of the result of the “automatically generated finding” and the result of the “radiographic interpretation finding”.

For example, when the degree of confidence of the “automatically generated finding” is low (equal to or smaller than threshold), and the result of the “automatically generated finding” is no lesion, there is a possibility that the AI analysis overlooked the lesion. When the user desires to confirm such lesion overlooked by the AI analysis, the combination of the result of the “automatically generated finding” and the result of the “radiographic interpretation finding” with the low degree of confidence in the “automatically generated finding” and the result of the “automatically generated finding” including no lesion can be displayed with highlight.

According to the present embodiment, according to user operation, the controller 31 as the setter is able to set to display with highlight the combination showing that the result of the “automatically generated finding” is the lesion is detected, and the result of the “radiographic interpretation finding” is the lesion is not detected among the combinations of the result of the “automatically generated finding” and the result of the “radiographic interpretation finding”.

Therefore, when the result of the “automatically generated finding” shows there is a lesion and the result of the “radiographic interpretation finding” shows there is no lesion, there is a possibility that the radiographic interpretation doctor who performs the radiographic interpretation overlooked the lesion, and such overlooking of the lesion can be avoided.

According to the present embodiment, according to user operation, the controller 31 as the setter is able to set to display with highlight the combination showing that the result of the “automatically generated finding” is the lesion is not detected and the result of the “radiographic interpretation finding” is the lesion is detected among the combinations of the result of the “automatically generated finding” and the result of the “radiographic interpretation finding”.

Therefore, when the result of the “automatically generated finding” shows there is no lesion and the result of the “radiographic interpretation finding” shows there is a lesion, there is a possibility that the AI analysis overlooked the lesion, and such overlooking of the lesion can be avoided.

According to the present embodiment, according to user operation, the controller 31 as the setter is able to set to display with highlight the combination showing that the result of the “automatically generated finding” is the lesion is not detected, and the result of the “radiographic interpretation finding” is the lesion is not detected among the combinations of the result of the “automatically generated finding” and the result of the “radiographic interpretation finding”.

Therefore, when the result of the “automatically generated finding” shows there is no lesion and the result of the “radiographic interpretation finding” shows there is no lesion, there is a possibility that both the AI analysis and the radiographic interpretation doctor who performs the radiographic interpretation overlooked the lesion, and such overlooking of the lesion can be avoided.

According to the present embodiment, according to user operation, the controller 31 as the setter is able to set to display with highlight the combination showing that the result of the “automatically generated finding” is the lesion is detected and the result of the “radiographic interpretation finding” is the lesion is detected among the combinations of the result of the “automatically generated finding” and the result of the “radiographic interpretation finding”.

Therefore, for example, when the degree of confidence of the “automatically generated finding” is low (equal to or smaller than the threshold), it is possible to confirm whether the AI analysis erroneously detected the lesion.

### [Modification]

Various embodiments of the present invention are described above but the present invention is not limited to the above embodiments, and various modifications are possible without leaving the scope of the present disclosure.

For example, according to the present embodiment, the medical information that is the target of analysis by the analysis apparatus 3 is the medical image. However, the medical information is not limited to the medical “image”.

The information obtained by various examinations on the patient may include a wide variety of medical information. Results obtained from various examinations such as electrocardiogram waveform data, phonocardiogram data, data regarding blood flow may also be included in the medical information.

According to the present embodiment, FIG. 1 illustrates each of the analysis apparatus 3, the radiographic interpretation terminal 4, and the image server 5 as separate apparatuses. However, the analysis apparatus 3 and the image server 4 or the analysis apparatus 3, the radiographic interpretation terminal 4, and the image server 5 may be configured as one apparatus or one system.

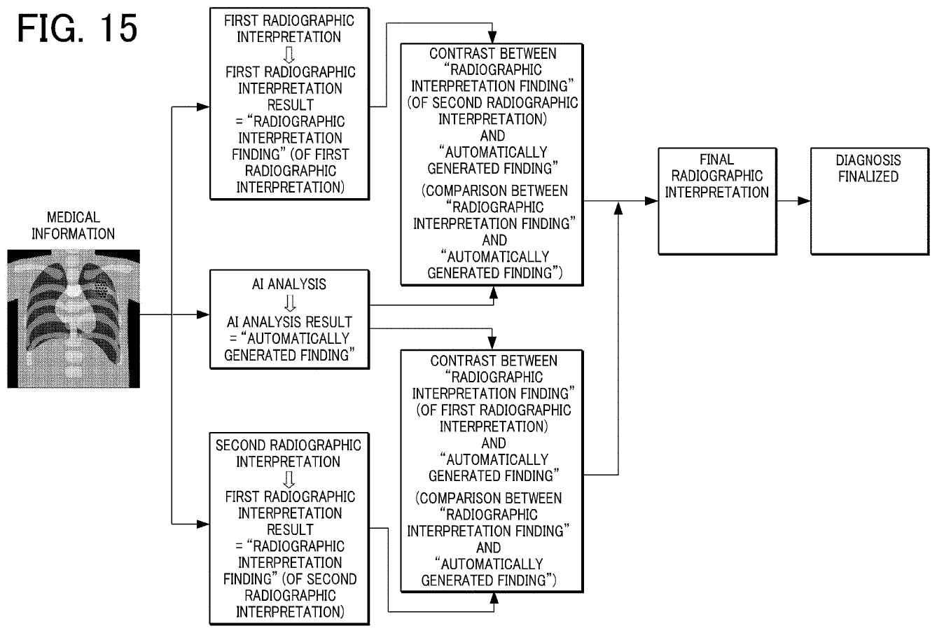

According to the present embodiment, the work flow shown in FIG. 4 is the work flow of the radiographic interpretation but the work flow is not limited to the above. The work flow of the radiographic interpretation may be the work flow as shown in FIG. 15.

According to the example shown in FIG. 15, the first radiographic interpretation, the second radiographic interpretation, and the AI analysis are performed in parallel, and the contrast (comparison) is performed between the “automatically generated finding” which is the result of the AI analysis and the “radiographic interpretation finding” of the first radiographic interpretation obtained by the first radiographic interpretation. Moreover, the contrast is performed between the “automatically generated finding” and the “radiographic interpretation finding” of the second radiographic interpretation obtained by the second radiographic interpretation. Then, the results of the above two contrasts are referred and the final radiographic interpretation is performed.

In this case, since the first radiographic interpretation and the second radiographic interpretation can be performed in parallel, different from the work flow shown in FIG. 4, the second radiographic interpretation doctor can start the radiographic interpretation without waiting for the result of the first radiographic interpretation.

Moreover, even if the results of the first radiographic interpretation and the second radiographic interpretation are different, each of the above can be compared with the result of the AI analysis. With this, the diagnosis result can be finalized.

Therefore, it is possible to shorten the time required to finalize the diagnosis.

When the first radiographic interpretation, the second radiographic interpretation and the AI analysis are performed in parallel, the “automatically generated finding”, the “radiographic interpretation finding” of the first radiographic interpretation finding and the “radiographic interpretation finding” of the second radiographic interpretation finding can be compared to each other and the result of the contrast can be obtained.

According to the present embodiment, as shown in FIG. 9 to FIG. 11, the controller 31 accepts setting of the condition of the highlight display of the examination information displayed on the work list display screen 361 on the condition setting screen 3625. That is, in the controller 31 as the setter, the user is able to set by user operation which combination to display with highlight among the combinations of the result of the “automatically generated finding” and the “radiographic interpretation finding”.

However, the method of setting the conditions for the highlight display is not limited to the illustrated examples shown in FIG. 9 to FIG. 11.

For example, as the conditions set for the highlight display, the controller 31 may accept whether or not to display with highlight the predetermined combination of the result of the “automatically generated finding” and the result of the “radiographic interpretation finding”. In this case, in the controller 31 as the setter, the user is able to set by user operation the combination to display with highlight among the combinations of the result of the “automatically generated finding” and the result of the “radiographic interpretation finding”.

The controller 31 according to the present embodiment controls the display 36 to perform the display with highlight based on the combination that is to be displayed with highlight set by the setter, but the present invention is not limited to the above. The configuration of the analysis apparatus 3 does not have to include the display 36, and in this case, the controller 31 controls the various displays provided in the radiographic interpretation terminal 4, the image server 5, and the external device which is not shown to perform the highlight display.

The present invention is not limited to the above described embodiments and modifications, and the embodiments and modifications can be suitably changed without leaving the scope of the present invention.

1. A radiographic interpretation management apparatus comprising:
a hardware processor,
wherein the hardware processor is configured to,
obtain an automatically generated finding obtained by computer processing on medical information,
obtain a radiographic interpretation finding created by a user based on the medical information, and
be able to set by user operation a combination that is to be displayed with highlight among combinations of a result of the automatically generated finding and a result of the radiographic interpretation finding.

2. The radiographic interpretation management apparatus according to claim 1, wherein the hardware processor is configured to be able to set by user operation which combination to display with highlight among the combinations of the result of the automatically generated finding and the result of the radiographic interpretation finding.

3. The radiographic interpretation management apparatus according to claim 1, wherein the result of the automatically generated finding includes a result of whether a lesion is detected by the automatically generated finding, and the result of the radiographic interpretation finding includes a result of whether a lesion is detected by the radiographic interpretation finding.

4. The radiographic interpretation management apparatus according to claim 1, wherein the result of the automatically generated finding includes a result of a detected position of a lesion detected by the automatically generated finding, and the result of the radiographic interpretation finding includes a result of a detected position of a lesion detected by the radiographic interpretation finding.

5. The radiographic interpretation management apparatus according to claim 1 further comprising a display that performs highlight display based on a combination in which setting to perform highlight display is set by the hardware processor.

6. The radiographic interpretation management apparatus according to claim 1, wherein the hardware processor is configured to be able to set by user operation a combination to be displayed with highlight among the combinations of the result of the automatically generated finding and the result of the radiographic interpretation finding and a threshold of a degree of confidence of the automatically generated finding, and
the radiographic interpretation management apparatus further comprises a display that performs highlight display of the combination of the result of the automatically generated finding and the result of the radiographic interpretation finding based on the combination in which the setting to perform highlight display is set by the hardware processor and the threshold of the degree of confidence of the automatically generated finding set by the hardware processor.

7. The radiographic interpretation management apparatus according to claim 6, wherein the display performs highlight display of the combination of the result of the automatically generated finding and the result of the radiographic interpretation finding when the degree of confidence of the automatically generated finding is equal to or lager than the threshold.

8. The radiographic interpretation management apparatus according to claim 6, wherein the display performs highlight display of the combination of the result of the automatically generated finding and the result of the radiographic interpretation finding when the degree of confidence of the automatically generated finding is equal to or smaller than the threshold.

9. The radiographic interpretation management apparatus according to claim 1, wherein the hardware processor is configured to be able to set by user operation to display with highlight the combination showing that the result of the automatically generated finding is a lesion is detected and the result of the radiographic interpretation finding is a lesion is not detected among the combinations of the result of the automatically generated finding and the result of the radiographic interpretation finding.

10. The radiographic interpretation management apparatus according to claim 1, wherein the hardware processor is configured to be able to set by user operation to display with highlight the combination showing that the result of the automatically generated finding is a lesion is not detected and the result of the radiographic interpretation finding is a lesion is detected among the combinations of the result of the automatically generated finding and the result of the radiographic interpretation finding.

11. The radiographic interpretation management apparatus according to claim 1, wherein the hardware processor is configured to be able to set by user operation to display with highlight the combination showing that the result of the automatically generated finding is a lesion is not detected and the result of the radiographic interpretation finding is a lesion is not detected among the combinations of the result of the automatically generated finding and the result of the radiographic interpretation finding.

12. The radiographic interpretation management apparatus according to claim 1, wherein the hardware processor is configured to be able to set by user operation to display with highlight the combination showing that the result of the automatically generated finding is a lesion is detected and the result of the radiographic interpretation finding is a lesion is detected among the combinations of the result of the automatically generated finding and the result of the radiographic interpretation finding.

13. A non-transitory computer-readable storage medium storing a program causing a computer to perform:
first obtaining to obtain an automatically generated finding obtained by computer processing on medical information,
second obtaining to obtain a radiographic interpretation finding created by a user based on the medical information, and
setting to be able to set by user operation a combination that is to be displayed with highlight among combinations of a result of the automatically generated finding and a result of the radiographic interpretation finding.

14. The storage medium including the program according to claim 13, wherein in the setting, the computer is able to set by user operation which combination to display with highlight among the combinations of the result of the automatically generated finding and the result of the radiographic interpretation finding.

15. The storage medium including the program according to claim 13, wherein the result of the automatically generated finding includes a result of whether a lesion is detected by the automatically generated finding, and the result of the radiographic interpretation finding includes a result of whether a lesion is detected by the radiographic interpretation finding.

16. The storage medium including the program according to claim 13, wherein the result of the automatically generated finding includes a result of a detected position of a lesion detected by the automatically generated finding, and the result of the radiographic interpretation finding includes a result of a detected position of a lesion detected by the radiographic interpretation finding.

17. The storage medium including the program according to claim 13 wherein the program allows the computer to control a display to perform highlight display based on a combination in which setting to perform highlight display is set.

18. The storage medium including the program according to claim 13, wherein the computer is able to set by user operation a combination to be displayed with highlight among the combinations of the result of the automatically generated finding and the result of the radiographic interpretation finding, and a threshold of a degree of confidence of the automatically generated finding, and
the computer allows the program to control the display to perform highlight display of the combination of the result of the automatically generated finding and the result of the radiographic interpretation finding based on the combination in which the setting to perform highlight display is set and the set threshold of the degree of confidence of the automatically generated finding.

19. The storage medium including the program according to claim 18, wherein the display is controlled to perform highlight display of the combination of the result of the automatically generated finding and the result of the radiographic interpretation finding when the degree of confidence of the automatically generated finding is equal to or lager than the threshold.

20. The storage medium including the program according to claim 18, wherein the display is controlled to perform highlight display of the combination of the result of the automatically generated finding and the result of the radiographic interpretation finding when the degree of confidence of the automatically generated finding is equal to or smaller than the threshold.

21. The storage medium including the program according to claim 13 wherein the computer is configured to be able to set by user operation to display with highlight the combination showing that the result of the automatically generated finding is a lesion is detected and the result of the radiographic interpretation finding is a lesion is not detected among the combinations of the result of the automatically generated finding and the result of the radiographic interpretation finding.

22. The storage medium including the program according to claim 13 wherein the computer is configured to be able to set by user operation to display with highlight the combination showing that the result of the automatically generated finding is a lesion is not detected and the result of the radiographic interpretation finding is a lesion is detected among the combinations of the result of the automatically generated finding and the result of the radiographic interpretation finding.

23. The storage medium including the program according to claim 13 wherein the computer is configured to be able to set by user operation to display with highlight the combination showing that the result of the automatically generated finding is a lesion is not detected and the result of the radiographic interpretation finding is a lesion is not detected among the combinations of the result of the automatically generated finding and the result of the radiographic interpretation finding.

24. The storage medium including the program according to claim 13 wherein the computer is configured to be able to set by user operation to display with highlight the combination showing that the result of the automatically generated finding is a lesion is detected and the result of the radiographic interpretation finding is a lesion is detected among the combinations of the result of the automatically generated finding and the result of the radiographic interpretation finding.

25. A radiographic interpretation management method comprising:
first obtaining to obtain an automatically generated finding obtained by computer processing on medical information,
second obtaining to obtain a radiographic interpretation finding created by a user based on the medical information, and
setting to be able to set by user operation a combination that is to be displayed with highlight among combinations of a result of the automatically generated finding and a result of the radiographic interpretation finding.
